(19) United States
(12) Patent Application Publication
(10) Pub. No.: US 2009/0029468 A1
(43) Pub. Date: Jan. 29, 2009

(54) ZINC FINGER BINDING DOMAINS FOR CNN

(75) Inventors: Carlos F. Barbas, III, Solana Beach, CA (US); Birgit Dreier, Regensdorf (CH)

Correspondence Address: SCHWEGMAN, LUNDBERG & WOESSNER, P.A., P.O. BOX 2938, MINNEAPOLIS, MN, 55402, US

(73) Assignee: The Scripps Research Institute, La Jolla, CA (US)

(21) Appl. No.: 12/118,601

(22) Filed: May 9, 2008

Related U.S. Application Data
(63) Continuation of application No. 10/487,268, filed on Jun. 18, 2004, now abandoned.
(60) Provisional application No. 60/313,864, filed on Aug. 21, 2001.
(60) Provisional application No. 60/313,693, filed on Aug. 20, 2001.

(51) Int. Cl. C12N 5/06; C07K 7/00; C07H 21/04

(52) U.S. Cl. 435/375; 530/329; 536/23.1

(57) ABSTRACT

Polypeptides that contain zinc finger-nucleotide binding regions that bind to nucleotide sequences of the formula CNN are provided. Compositions containing a plurality of polypeptides, polynucleotides that encode such polypeptides and methods of regulating gene expression with such polypeptides, compositions and polynucleotide's are also provided.

### CROSS REFERENCE TO RELATED APPLICATIONS

This application is a continuation-in-part of U.S. Provisional Patent Applications Ser. Nos. 60/313,864 and 60/313,693, filed Aug. 20, 2001, the disclosures of which are incorporated herein by reference.

Funds used to support some of the studies reported herein were provided by the National Institutes of Health (NIH GM 53910). The United States Government, therefore, may have certain rights in the invention.

### TECHNICAL FIELD OF THE INVENTION

The field of this invention is zinc finger protein binding to target nucleotides. More particularly, the present invention pertains to amino acid residue sequences within the α-helical domain of zinc fingers that specifically bind to target nucleotides of the formula 5′-(CNN)-3′.

### BACKGROUND OF THE INVENTION

The construction of artificial transcription factors has been of great interest in the past years. Gene expression can be specifically regulated by polydactyl zinc finger proteins fused to regulatory domains. Zinc finger domains of the Cys₂-His₂ family have been most promising for the construction of artificial transcription factors due to their modular structure. Each domain consists of approximately 30 amino acids and folds into a α-structure stabilized by hydrophobic interactions and chelation of a zinc ion by the conserved Cys₂-His₂ residues. To date, the best characterized protein of this family of zinc finger proteins is the mouse transcription factor Zif 268 [Pavletich et al., (1991) Science 252(5007), 809-817; Elrod-Erickson et al., (1996) Structure 4(10), 1171-1180]. The analysis of the Zif 268/DNA complex suggested that DNA binding is predominantly achieved by the interaction of amino acid residues of the α-helix in position −1, 3, and 6 with the 3′, middle, and 5′ nucleotide of a 3 bp DNA subsite, respectively. Positions 1, 2 and 5 have been shown to make direct or water-mediated contacts with the phosphate backbone of the DNA. Leucine is usually found in position 4 and packs into the hydrophobic core of the domain. Position 2 of the α-helix has been shown to interact with other helix residues and, in addition, can make contact to a nucleotide outside the 3 bp subsite [Pavletich et al., (1991) Science 252(5007), 809-817; Elrod-Erickson et al., (1996) Structure 4(10), 1171-1180; Isalan, M. et al., (1997) Proc Natl Acad Sci USA 94(11), 5617-5621].

The selection of modular zinc finger domains recognizing each of the 5′-GNN-3′ DNA subsites with high specificity and affinity and their refinement by site-directed mutagenesis has been demonstrated (U.S. Pat. No. 6,140,081, the disclosure of which is incorporated herein by reference). These modular domains can be assembled into zinc finger proteins recognizing extended 18 bp DNA sequences which are unique within the human or any other genome. In addition, these proteins function as transcription factors and are capable of altering gene expression when fused to regulatory domains and can even be made hormone-dependent by fusion to ligand-binding domains of nuclear hormone receptors. To allow the rapid construction of zinc finger-based transcription factors binding to any DNA sequence it is important to extend the existing set of modular zinc finger domains to recognize each of the 64 possible DNA triplets. This aim can be achieved by phage display selection and/or rational design. Due to the limited structural data on zinc finger/DNA interaction, rational design of zinc proteins is very time-consuming and may not be possible in many instances. In addition, most naturally occurring zinc finger proteins consist of domains recognizing the 5′-(GNN)-3′ type of DNA sequences. The most promising approach to identify novel zinc finger domains binding to DNA target sequences of the type 5′-NNN-3′ is selection via phage display. The limiting step for this approach is the construction of libraries that allow the specification of a 5′ adenine, cytosine or thymine. Phage display selections have been based on Zif268 in which different fingers of this protein were randomized [Choo et al., (1994) Proc. Natl. Acad. Sci. U.S.A. 91(23), 11168-72; Rebar et al., (1994) Science (Washington, D.C., 1883—) 263(5147), 671-3; Jamieson et al., (1994) Biochemistry 33, 5689-5695; Wu et al., (1995) PNAS 92, 344-348; Jamieson et al., (1996) Proc Natl Acad Sci USA 93, 12834-12839; Greisman et al., (1997) Science 275(5300), 657-661]. A set of 16 domains recognizing the 5′-GNN-3′ type of DNA sequences has previously been reported from a library where finger 2 of C7, a derivative of Zif268 [Wu et al., (1995) PNAS 92, 344-348 Wu, 1995], was randomized [Segal et al., (1999) Proc Natl Acad Sci US A 96(6), 2758-2763]. In such a strategy, selection is limited to domains recognizing 5′-GNN-3′ or 5′-TNN-3′ due to the Asp² of finger 3 making contact with the complementary base of a 5′ guanine or thymine in the finger-2 subsite [Pavletich et al., (1991) Science 252(5007), 809-817; Elrod-Erickson et al., (1996) Structure 4(10), 1171-1180].

The present approach is based on the modularity of zinc finger domains that allows the rapid construction of zinc finger proteins by the scientific community and demonstrates that the concerns regarding limitation imposed by cross-subsite interactions only occurs in a limited number of cases. The present disclosure introduces a new strategy for selection of zinc finger domains specifically recognizing the 5′-CNN-3′ type of DNA sequences. Specific DNA-binding properties of these domains was evaluated by a multi-target BLISA against all sixteen 5′-CNN-3′ triplets. These domains can be readily incorporated into polydactyl proteins containing various numbers of 5′-CNN-3′ domains, each specifically recognizing extended 18 bp sequences. Furthermore, these domains can specifically alter gene expression when fused to regulatory domains. These results underline the feasibility of constructing polydactyl proteins from pre-defined building blocks. In addition, the domains characterized here greatly increase the number of DNA sequences that can be targeted with artificial transcription factors.

### BRIEF SUMMARY OF THE INVENTION

In one aspect, the present invention provides an isolated and purified zinc finger nucleotide binding polypeptide that contains a nucleotide binding region of from 5 to 10 amino acid residues, which region binds preferentially to a target nucleotide of the formula CNN, where N is A, C, G or T. Preferably, the target nucleotide has the formula CAA, CAC, CAG, CAT, CCA, CCC, CCG, CCT, CGA, CGC, CGG, CGT, CTA, CTC, CTG or CTT. In one embodiment, a polypeptide of the invention contains a binding region that has an amino acid residue sequence with the same nucleotide binding characteristics as any of SEQ ID NOs:1-25. Such a polypeptide competes for binding to a nucleotide target with any of SEQ ID NOs:1-25. Preferably, the binding region has the amino acid residue sequence of any of SEQ ID NOs:1-25. In one embodiment, this invention provides an isolated and purified zinc finger nucleotide binding polypeptide consisting of an amino acid residue sequence of any of SEQ ID NOs:1-25.

In another aspect, the present invention provides a peptide composition that contains a plurality of and, preferably from about 2 to about 12 of a zinc finger nucleotide binding polypeptide as disclosed herein. The polypeptides are operatively linked such as linked via a flexible peptide linker of from 5 to 15 amino acid residues. Operatively linked preferably occurs via a flexible peptide linker such as that shown in SEQ ID NO:30. Such a composition binds to a nucleotide sequence that contains a sequence of the formula 5′ (CNN)ₙ-3′, where N is A, C, G or T and n is 2 to 12. Preferably, the composition contains from about 2 to about 6 zinc finger nucleotide binding polypeptides and binds to a nucleotide sequence that contains a sequence of the formula 5′-(CNN)ₙ-3′, where n is 2 to 6. Binding occurs with a K_{D} of from 1 fM to 10 μM. Preferably binding occurs with a K_{D} of from 10 fM to 1 μM, from 10 pM to 100 nM, from 100 pM to 10 nM and, more preferably with a K_{D} of from 1 nM to 10 nM. In preferred embodiments, both a polypeptide and a composition of this invention are operatively linked to one or more transcription regulating factors such as a repressor of transcription or an activator of transcription.

The present invention further provides polynucleotides that encode a polypeptide or a composition of this invention, expression vectors that contain such polynucleotides and host cells transformed with the polynucleotide or expression vector.

The present invention further provides a process of regulating expression of a nucleotide sequence that contains the target nucleotide sequence 5′-(CNN)-3′. The target nucleotide sequence can be located anywhere within a longer 5′-(NNN)-3′ sequence. The process includes the step of exposing the nucleotide sequence to an effective amount of a zinc finger nucleotide binding polypeptide or composition as set forth herein. In one embodiment, a process regulates expression of a nucleotide sequence that contains the sequence 5′-(CNN)ₙ-3′, where n is 2 to 12. The process includes the step of exposing the nucleotide sequence to an effective amount of a composition of this invention. The sequence 5′-(CNN)ₙ-3 can be located in the transcribed region of the nucleotide sequence, in a promotor region of the nucleotide sequence, or within an expressed sequence tag. The composition is preferably operatively linked to one or more transcription regulating factors such as a repressor of transcription or an activator of transcription. In one embodiment, the nucleotide sequence is a gene such as a eukaryotic gene, a prokaryotic gene or a viral gene. The eukaryotic gene can be a mammalian gene such as a human gene or a plant gene. The prokaryotic gene can be a bacterial gene.

### BRIEF DESCRIPTION OF THE DRAWINGS

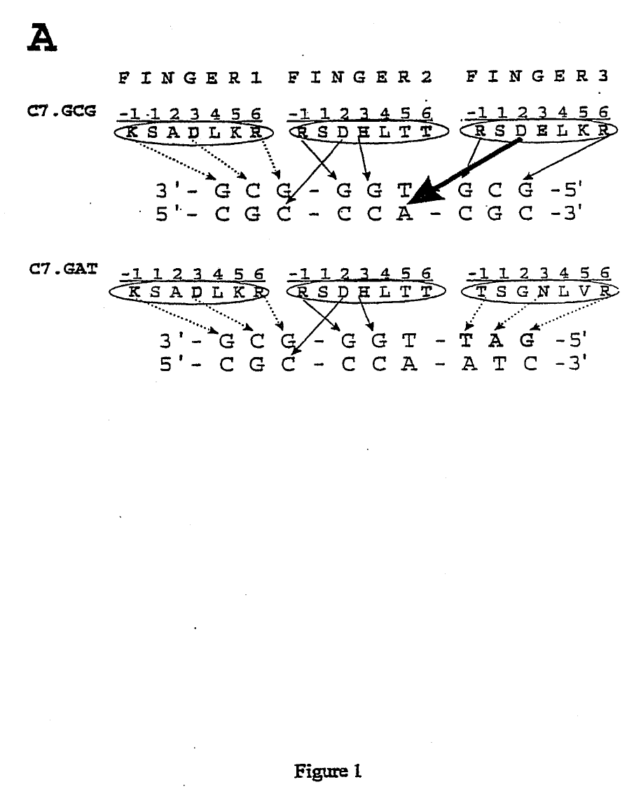

In the drawings that form a portion of the specification, FIG. 1 shows, in two panels designated 1A and 1B, schematically, construction of the zinc finger phage display library (A) and multitarget specificity ELISA for the C7 proteins (B).

### DETAILED DESCRIPTION OF THE INVENTION

### Definitions

Unless defined otherwise, all technical and scientific terms used herein have the same meaning as is commonly understood by one of skill in the art to which this invention belongs.

As used herein, the transcription regulating domain or factor refers to the portion of the fusion polypeptide provided herein that functions to regulate gene transcription. Exemplary and preferred transcription repressor domains are ERD, KRAB, SID, Deacetylase, and derivatives, multimers and combinations thereof such as KRAB-ERD, SID-ERD, (KRAB)₂, (KRAB)₃, KRAB-A, (KRAB-A)₂, (SID)₂, (KRAB-A)-SID and SD-(KRAB-A). As used herein, nucleotide binding domain or region, refers to the portion of a polypeptide or composition provided herein that provides specific nucleic acid binding capability. The nucleotide binding region functions to target a subject polypeptide to specific genes. As used herein, operatively linked means that elements of a polypeptide, for example, are linked such that each perform or functions as intended. For example, a repressor is attached to the binding domain in such a manner that, when bound to a target nucleotide via that binding domain, the repressor acts to inhibit or prevent transcription. Linkage between and among elements may be direct or indirect, such as via a linker. The elements are not necessarily adjacent. Hence a repressor domain can be linked to a nucleotide binding domain using any linking procedure well known in the art. It may be necessary to include a linker moiety between the two domains. Such a linker moiety is typically a short sequence of amino acid residues that provides spacing between the domains. So long as the linker does not interfere with any of the functions of the binding or repressor domains, any sequence can be used.

As used herein, “modulating” envisions the inhibition or suppression of expression from a promoter containing a zinc finger-nucleotide binding motif when it is over-activated, or augmentation or enhancement of expression from such a promoter when it is underactivated.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p. 224).

As used herein, “expression vector” refers to a plasmid, virus or other vehicle known in the art that has been manipulated by insertion or incorporation of heterologous DNA, such as nucleic acid encoding the fusion proteins herein or expression cassettes provided herein. Such expression vectors contain a promotor sequence for efficient transcription of the inserted nucleic acid in a cell. The expression vector typically contains an origin of replication, a promoter, as well as specific genes that permit phenotypic selection of transformed cells.

As used herein, “host cells” are cells in which a vector can be propagated and its DNA expressed. The term also includes any progeny of the subject host cell. It is understood that all progeny may not be identical to the parental cell since there may be mutations that occur during replication. Such progeny are included when the term “host cell” is used. Methods of stable transfer where the foreign DNA is continuously maintained in the host are known in the art.

As used herein, genetic therapy involves the transfer of heterologous DNA to the certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, or it may encode a product, such as a peptide or RNA that in some manner mediates, directly or indirectly, expression of a therapeutic product. Genetic therapy may also be used to deliver nucleic acid encoding a gene product that replaces a defective gene or supplements a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof. Genetic therapy may also involve delivery of an inhibitor or repressor or other modulator of gene expression.

As used herein, heterologous DNA is DNA that encodes RNA and proteins that are not normally produced in vivo by the cell in which it is expressed or that mediates or encodes mediators that alter expression of endogenous DNA by affecting transcription translation, or other regulatable biochemical processes. Heterologous DNA may also be referred to as foreign DNA. Any DNA that one of skill in the art would recognize or consider as heterologous or foreign to the cell in which is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

Hence, herein heterologous DNA or foreign DNA, includes a DNA molecule not present in the exact orientation and position as the counterpart DNA molecule found in the genome. It may also refer to a DNA molecule from another organism or species (i.e., exogenous).

As used herein, a therapeutically effective product is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease. Typically, DNA encoding a desired gene product is cloned into a plasmid vector and introduced by routine methods, such as calcium-phosphate mediated DNA uptake (see, (1981) Somat. Cell. Mol. Genet. 7:603-616) or microinjection, into producer cells, such as packaging cells. After amplification in producer cells, the vectors that contain the heterologous DNA are introduced into selected target cells.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors”. Also included are vectors that allow cloning of cDNA (complementary DNA) from mRNAs produced using reverse transcriptase. As used herein, a gene refers to a nucleic acid molecule whose nucleotide sequence encodes an RNA or polypeptide. A gene can be either RNA or DNA. Genes may include regions preceding and following the coding region (leader and trailer) as well as intervening sequences (introns) between individual coding segments (exons).

As used herein, isolated with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated”, but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” the nucleic acid is free of the coding sequences of those genes that, in a naturally-occurring genome immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, “modulate” refers to the suppression, enhancement or induction of a function. For example, zinc finger-nucleic acid binding domains and variants thereof may modulate a promoter sequence by binding to a motif within the promoter, thereby enhancing or suppressing transcription of a gene operatively linked to the promoter cellular nucleotide sequence. Alternatively, modulation may include inhibition of transcription of a gene where the zinc finger-nucleotide binding polypeptide variant binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript.

As used herein, “inhibit” refers to the suppression of the level of activation of transcription of a structural gene operably linked to a promoter. For example, for the methods herein the gene includes a zinc finger-nucleotide binding motif.

As used herein, a transcriptional regulatory region refers to a region that drives gene expression in the target cell. Transcriptional regulatory regions suitable for use herein include but are not limited to the human cytomegalovirus (CMV) immediate-early enhancer/promoter, the SV40 early enhancer/promoter, the JC polyomavirus promoter, the albumin promoter, PGK and the α-actin promoter coupled to the CMV enhancer.

As used herein, a promoter region of a gene includes the regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or, for example, an onco-promoter. An onco-promoter is generally a virus-derived promoter. Viral promoters to which zinc finger binding polypeptides may be targeted include, but are not limited to, retroviral long terminal repeats (LTRs), and Lentivirus promoters, such as promoters from human T-cell lymphotrophic virus (HTLV) 1 and 2 and human immunodeficiency virus (HIV) 1 or 2.

As used herein, “effective amount” includes that amount that results in the deactivation of a previously activated promoter or that amount that results in the inactivation of a promoter containing a zinc finger-nucleotide binding motif, or that amount that blocks transcription of a structural gene or translation of RNA. The amount of zinc finger derived-nucleotide binding polypeptide required is that amount necessary to either displace a native zinc finger-nucleotide binding protein in an existing protein/promoter complex, or that amount necessary to compete with the native zinc finger-nucleotide binding protein to form a complex with the promoter itself. Similarly, the amount required to block a structural gene or RNA is that amount which binds to and blocks RNA polymerase from reading through on the gene or that amount which inhibits translation, respectively. Preferably, the method is performed intracellularly. By functionally inactivating a promoter or structural gene, transcription or translation is suppressed. Delivery of an effective amount of the inhibitory protein for binding to or “contacting” the cellular nucleotide sequence containing the zinc finger-nucleotide binding protein motif, can be accomplished by one of the mechanisms described herein, such as by retroviral vectors or liposomes, or other methods well known in the art.

As used herein, “truncated” refers to a zinc finger-nucleotide binding polypeptide derivative that contains less than the full number of zinc fingers found in the native zinc finger binding protein or that has been deleted of non-desired sequences. For example, truncation of the zinc finger-nucleotide binding protein TFIIIA, which naturally contains nine zinc fingers, might be a polypeptide with only zinc fingers one through three. Expansion refers to a zinc finger polypeptide to which additional zinc finger modules have been added. For example, TFIIIA may be extended to 12 fingers by adding 3 zinc finger domains. In addition, a truncated zinc finger-nucleotide binding polypeptide may include zinc finger modules from more than one wild type polypeptide, thus resulting in a “hybrid” zinc finger-nucleotide binding polypeptide.

As used herein, “mutagenized” refers to a zinc finger derived-nucleotide binding polypeptide that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated zinc finger-nucleotide binding proteins can also be mutagenized.

As used herein, a polypeptide “variant” or “derivative” refers to a polypeptide that is a mutagenized form of a polypeptide or one produced through recombination but that still retains a desired activity, such as the ability to bind to a ligand or a nucleic acid molecule or to modulate transcription.

As used herein, a zinc finger-nucleotide binding polypeptide “variant” or “derivative” refers to a polypeptide that is a mutagenized form of a zinc finger protein or one produced through recombination. A variant may be a hybrid that contains zinc finger domain(s) from one protein linked to zinc finger domain(s) of a second protein, for example. The domains may be wild type or mutagenized. A “variant” or “derivative” includes a truncated form of a wild type zinc finger protein, which contains less than the original number of fingers in the wild type protein. Examples of zinc finger-nucleotide binding polypeptides from which a derivative or variant may be produced include TFIIIA and zif268. Similar terms are used to refer to “variant” or “derivative” nuclear hormone receptors and “variant” or “derivative” transcription effector domains.

As used herein a “zinc finger-nucleotide binding target or motif” refers to any two or three-dimensional feature of a nucleotide segment to which a zinc finger-nucleotide binding derivative polypeptide binds with specificity. Included within this definition are nucleotide sequences, generally of five nucleotides or less, as well as the three dimensional aspects of the DNA double helix, such as, but are not limited to, the major and minor grooves and the face of the helix. The motif is typically any sequence of suitable length to which the zinc finger polypeptide can bind. For example, a three finger polypeptide binds to a motif typically having about 9 to about 14 base pairs. Preferably, the recognition sequence is at least about 16 base pairs to ensure specificity within the genome. Therefore, zinc finger-nucleotide binding polypeptides of any specificity are provided. The zinc finger binding motif can be any sequence designed empirically or to which the zinc finger protein binds. The motif may be found in any DNA or RNA sequence, including regulatory sequences, exons, introns, or any non-coding sequence.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the composition.

As used herein, the term “vector” refers to a nucleic acid molecule capable of transporting between different genetic environments another nucleic acid to which it has been operatively linked. Preferred vectors are those capable of autonomous replication and expression of structural gene products present in the DNA segments to which they are operatively linked. Vectors, therefore, preferably contain the replicons and selectable markers described earlier.

As used herein with regard to nucleic acid molecules, including DNA fragments, the phrase “operatively linked” means the sequences or segments have been covalently joined, preferably by conventional phosphodiester bonds, into one strand of DNA, whether in single or double-stranded form such that operatively linked portions functions as intended. The choice of vector to which transcription unit or a cassette provided herein is operatively linked depends directly, as is well known in the art, on the functional properties desired, e.g., vector replication and protein expression, and the host cell to be transformed, these being limitations inherent in the art of constructing recombinant DNA molecules.

As used herein, administration of a therapeutic composition can be effected by any means, and includes, but is not limited to, subcutaneous, intravenous, intramuscular, intrasternal, infusion techniques, intraperitoneally administration and parenteral administration.

### I. THE INVENTION

The present invention provides zinc finger-nucleotide binding polypeptides, compositions containing one or more such polypeptides, polynucleotides that encode such polypeptides and compositions, expression vectors containing such polynucleotides, cells transformed with such polynucleotides or expression vectors and the use of the polypeptides, compositions, polynucleotides and expression vectors for modulating nucleotide structure and/or function.

### II. POLYPEPTIDES

The present invention provides an isolated and purified zinc finger nucleotide binding polypeptide. The polypeptide contains a nucleotide binding region of from 5 to 10 amino acid residues and, preferably about 7 amino acid residues. The nucleotide binding region binds preferentially to a target nucleotide of the formula CNN, where N is A, C, G or T. Preferably, the target nucleotide has the formula CAA, CAC, CAG, CAT, CCA, CCC, CCG, CCT, CGA, CGC, CGG, CGT, CTA, CTC, CTG or CTT.

A polypeptide of this invention is non-naturally occurring variant. As used herein, the term “non-naturally occurring” means, for example, one or more of the following: (a) a peptide comprised of a non-naturally occurring amino acid sequence; (b) a peptide having a non-naturally occurring secondary structure not associated with the peptide as it occurs in nature; (c) a peptide which includes one or more amino acids not normally associated with the species of organism in which that peptide occurs in nature; (d) a peptide which includes a stereoisomer of one or more of the amino acids comprising the peptide, which stereoisomer is not associated with the peptide as it occurs in nature; (e) a peptide which includes one or more chemical moieties other than one of the natural amino acids; or (1) an isolated portion of a naturally occurring amino acid sequence (e.g., a truncated sequence). A polypeptide of this invention exists in an isolated form and purified to be substantially free of contaminating substances. A polypeptide is synthetic in nature. That is, the polypeptide is isolated and purified from natural sources or made de novo using techniques well known in the art. A zinc finger-nucleotide binding polypeptide refers to a polypeptide that is, preferably, a mutagenized form of a zinc finger protein or one produced through recombination. A polypeptide may be a hybrid which contains zinc finger domain(s) from one protein linked to zinc finger domain(s) of a second protein, for example. The domains may be wild type or mutagenized. A polypeptide includes a truncated form of a wild type zinc finger protein. Examples of zinc finger proteins from which a polypeptide can be produced include TFIIIA and zif268.

A zinc finger-nucleotide binding polypeptide of this invention comprises a unique heptamer (contiguous sequence of 7 amino acid residues) within the α-helical domain of the polypeptide, which heptameric sequence determines binding specificity to a target nucleotide. That heptameric sequence can be located anywhere within the α-helical domain but it is preferred that the heptamer extend from position −1 to position 6 as the residues are conventionally numbered in the art. A polypeptide of this invention can include any P-sheet and framework sequences known in the art to function as part of a zinc finger protein. A large number of zinc finger-nucleotide binding polypeptides were made and tested for binding specificity against target nucleotides containing a CNN triplet.

The zinc finger-nucleotide binding polypeptide derivative can be derived or produced from a wild type zinc finger protein by truncation or expansion, or as a variant of the wild type-derived polypeptide by a process of site directed mutagenesis, or by a combination of the procedures. The term “truncated” refers to a zinc finger-nucleotide binding polypeptide that contains less that the full number of zinc fingers found in the native zinc finger binding protein or that has been deleted of non-desired sequences. For example, truncation of the zinc finger-nucleotide binding protein TFIIIA, which naturally contains nine zinc fingers, might be a polypeptide with only zinc fingers one through three. Expansion refers to a zinc finger polypeptide to which additional zinc finger modules have been added. For example, TFIIIA may be extended to 12 fingers by adding 3 zinc finger domains. In addition, a truncated zinc finger-nucleotide binding polypeptide may include zinc finger modules from more than one wild type polypeptide, thus resulting in a “hybrid” zinc finger-nucleotide binding polypeptide.

The term “mutagenized” refers to a zinc finger derived-nucleotide binding polypeptide that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated zinc finger-nucleotide binding proteins can also be mutagenized. Examples of known zinc finger-nucleotide binding polypeptides that can be truncated, expanded, and/or mutagenized according to the present invention in order to inhibit the function of a nucleotide sequence containing a zinc finger-nucleotide binding motif includes TFIIIA and zif268. Those of skill in the art know other zinc finger-nucleotide binding proteins.

In one embodiment, a polypeptide of the invention contains a binding region that has an amino acid residue sequence with the same nucleotide binding characteristics as any of SEQ ID NOs:1-25. A detailed description of how those binding characteristics were determined can be found hereinafter in the Examples. Such a polypeptide competes for binding to a nucleotide target with any of SEQ ID NOs:1-25. That is, a preferred polypeptide contains a binding region that will displace, in a competitive manner, the binding of any of SEQ IDS NOs:1-25. Means for determining competitive binding are well known in the art. Preferably, the binding region has the amino acid residue sequence of any of SEQ ID NOs:1-25.

A polypeptide of this invention can be made using a variety of standard techniques well known in the art. As disclosed in detail hereinafter in the Examples, phage display libraries of zinc finger proteins were created and selected under conditions that favored enrichment of sequence specific proteins. Zinc finger domains recognizing a number of sequences required refinement by site-directed mutagenesis that was guided by both phage selection data and structural information.

Previously we reported the characterization of 16 zinc finger domains specifically recognizing each of the 5′-GNN-3′ type of DNA sequences, that were isolated by phage display selections based on C7, a variant of the mouse transcription factor Zif268 and refined by site-directed mutagenesis [Segal et al., (1999) Proc Natl Acad Sci USA 96(6), 2758-2763; Dreier et al., (2000) J. Mol. Biol. 303, 489-502; and U.S. Pat. No. 6,140,081, the disclosure of which is incorporated herein by reference]. In general, the specific DNA recognition of zinc finger domains of the Cys₂-His₂ type is mediated by the amino acid residues—1, 3, and 6 of each α-helix, although not in every case are all three residues contacting a DNA base. One dominant cross-subsite interaction has been observed from position 2 of the recognition helix. Asp² has been shown to stabilize the binding of zinc finger domains by directly contacting the complementary adenine or cytosine of the 5′ thymine or guanine, respectively, of the following 3 bp subsite. These non-modular interactions have been described as target site overlap. In addition, other interactions of amino acids with nucleotides outside the 3 bp subsites creating extended binding sites have been reported [Pavletich et al., (1991) Science 252(5007), 809-817; Elrod-Erickson et al., (1996) Structure 4(10), 1171-1180; Isalan et al., (1997) Proc Natl Acad Sci USA 94(11), 5617-5621].

Selection of the previously reported phage display library for zinc finger domains binding to 5′ nucleotides other than guanine or thymine met with no success, due to the cross-subsite interaction from aspartate in position 2 of the finger-3 recognition helix RSD-E-LKR (SEQ ID NO:26), (FIG. 1). To extend the availability of zinc finger domains for the construction of artificial transcription factors, domains specifically recognizing the 5′-ANN-3′ type of DNA sequences were selected (U.S. patent application Ser. No. 09/791,106, filed Feb. 21, 2001, the disclosure of which is incorporated herein by reference). Other groups have described a sequential selection method which led to the characterization of domains recognizing four 5′-ANN-3′ subsites, 5′-AAA-3′,5′-AAG-3′, 5′-ACA3′, and 5′-ATA-3′ [Greisman et al., (1997) Science 275(5300), 657-661; Wolfe et al., (1999) J Mol Biol 285(5), 1917-1934]. The present disclosure uses an approach to select zinc finger domains recognizing CNN sites by eliminating the target site overlap. First, finger 3 of C7 (RSD-E-RKR) (SEQ ID NO:27) binding to the subsite 5′-GCG-3′ was exchanged with a domain which did not contain aspartate in position 2 (FIG. 1). The helix TSG-N-LVR (SEQ ID NO:28), previously characterized in finger 2 position to bind with high specificity to the triplet 5′-GAT-3′, seemed a good candidate. This 3-finger protein (C7.GAT; FIG. 1A, lower panel), containing finger 1 and 2 of C7 and the 5′-GAT-3′-recognition helix in finger-3 position, was analyzed for DNA-binding specificity on targets with different finger-2 subsites by multi-target ELISA in comparison with the original C7 protein (C7.GCG; FIG. 1B). Both proteins bound to the 5′-TGG-3′ subsite (note that C7.GCG binds also to 5′-GGG-3′ due to the 5′ specification of thymine or guanine by Asp² of finger 3 which has been reported earlier. The recognition of the 5′ nucleotide of the finger-2 subsite was evaluated using a mixture of all 16 5′-XNN-3′ target sites (X=adenine, guanine, cytosine orthymine). Indeed, while the original C7.GCG protein specified a guanine or thymine in the 5′ position of finger 2, C7.GAT did not specify a base, indicating that the cross-subsite interaction to the adenine complementary to the 5′ thymine was abolished. A similar effect has previously been reported for variants of Zif268 where Asp² was replaced by Ala² by site-directed mutagenesis [Isalan et al., (1997) Proc Natl Acad Sci USA 94(11), 5617-5621; Dreier et al., (2000) J. Mol. Biol. 303, 489-502]. The affinity of C7.GAT, measured by gel mobility shift analysis, was found to be relative low, about 400 nM compared to 0.5 nM for C7.GCG [Segal et al., (1999) Proc Natl Acad Sci USA 96(6), 2758-2763], which may in part be due to the lack of the Asp² in finger 3.

Based on the 3-finger protein C7.GAT, a library was constructed in the phage display vector pComb3H [Barbas et al., (1991) Proc. Natl. Acad. Sci. USA 88, 7978-7982; Rader et al., (1997) Curr. Opin. Biotechnol. 8(4), 503-508]. Randomization involved positions −1, 1, 2, 3, 5, and 6 of the α-helix of finger 2 using a VNS codon doping strategy (V=adenine, cytosine or guanine, N=adenine, cytosine, guanine or thymine, S=cytosine or guanine). This allowed 24 possibilities for each randomized amino acid position, whereas the aromatic amino acids Trp, Phe, and Tyr, as well as stop codons, were excluded in this strategy. Because Leu is predominately found in position 4 of the recognition helices of zinc finger domains of the type Cys₂-His₂ this position was not randomized. After transformation of the library into ER2537 cells (New England Biolabs) the library contained 1.5×10⁹ members. This exceeded the necessary library size by 60-fold and was sufficient to contain all amino acid combinations.

Six rounds of selection of zinc finger-displaying phage were performed binding to each of the sixteen 5′-GAT-CNN-GCG-3′ (SEQ ID NO:29) biotinylated hairpin target oligonucleotides, respectively, in the presence of non-biotinylated competitor DNA. Stringency of the selection was increased in each round by decreasing the amount of biotinylated target oligonucleotide and increasing amounts of the competitor oligonucleotide mixtures. In the sixth round the target concentration was usually 18 nM, 5′-ANN-3′,5′-GNN-3′, and 5′-TNN-3′ competitor mixtures were in 5-fold excess for each oligonucleotide pool, respectively, and the specific 5′-CNN-3′ mixture (excluding the target sequence) in 10-fold excess. Phage binding to the biotinylated target oligonucleotide was recovered by capture to streptavidin-coated magnetic beads. Clones were usually analyzed after the sixth round of selection.

### III. COMPOSITIONS

In another aspect, the present invention provides a plurality of zinc finger-nucleotide binding polypeptides operatively linked in such a manner to specifically bind a nucleotide target motif defined as 5′-(CNN)ₙ-3′, where n is an integer greater than 1. The target motif can be located within any longer nucleotide sequence (e.g., from 3 to 13 or more TNN, GNN, ANN or NNN sequences). Preferably, n is an integer from 2 to about 12, and more preferably from 2 to 6. The individual polypeptides are preferably linked with oligopeptide linkers. Such linkers preferably resemble a linker found in naturally occurring zinc finger proteins. A preferred linker for use in the present invention is the amino acid residue sequence TGEKP (SEQ ID NO:30). Other linkers such as glycine or serine repeats are well known in the art to link peptides (e.g., single chain antibody domains) and can be used in a composition of this invention.

A polypeptide or composition of this invention can be operatively linked to one or more functional peptides. Such functional peptides are well known in the art and can be a transcription regulating factor such as a repressor or activation domain or a peptide having other functions. Exemplary and preferred such functional peptides are nucleases, methylases, nuclear localization domains, and restriction enzymes such as endo- or ectonucleases (See, e.g., Chandrasegaran and Smith, Biol. Chem., 380:841-848, 1999).

An exemplary repression domain peptide is the ERF repressor domain (ERD) (Sgouras, D. N., Athanasiou, M. A., Beal, G. J., Jr., Fisher, R. J., Blair, D. G. & Mavrothalassitis, G. J. (1995) EMBO J. 14, 4781-4793), defined by amino acids 473 to 530 of the ets2 repressor factor (ERF). This domain mediates the antagonistic effect of ERF on the activity of transcription factors of the ets family. A synthetic repressor is constructed by fusion of this domain to the N- or C-terminus of the zinc finger protein. A second repressor protein is prepared using the Krüppel-associated box (KRAB) domain (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). This repressor domain is commonly found at the N-terminus of zinc finger proteins and presumably exerts its repressive activity on TATA-dependent transcription in a distance- and orientation-independent manner (Pengue, G. & Lania, L. (1996) Proc. Natl. Acad. Sci. USA 93, 1015-1020), by interacting with the RING finger protein KAP-1 (Friedman, J. R., Fredericks, W. J., Jensen, D. E., Speicher, D. W., Huang, X.-P., Neilson, E. G. & Rauscher III, F. J. (1996) Genes & Dev. 10, 2067-2078). We utilized the KRA-3 domain found between amino acids 1 and 97 of the zinc finger protein KOX1 (Margolin, J. F., Friedman, J. R., Meyer, W., K.-H., Vissing, H., Thiesen, H.-J. & Rauscher III, P. J. (1994) Proc. Natl. Acad. Sci. USA 91, 4509-4513). In this case an N-terminal fusion with a zinc-finger polypeptide is constructed. Finally, to explore the utility of histone deacetylation for repression, amino acids 1 to 36 of the Mad mSIN3 interaction domain (SID) are fused to the N-terminus of the zinc finger protein (Ayer, D. E., Laherty, C. D., Lawrence, Q. A., Armstrong, A. P. & Eisenman, R. N. (1996) Mol. Cell. Biol. 16, 5772-5781). This small domain is found at the N-terminus of the transcription factor Mad and is responsible for mediating its transcriptional repression by interacting with mSIN3, which in turn interacts the co-repressor N-CoR and with the histone deacetylase mRPD1 (Heinzel, T., Lavinsky, R. M., Mullen, T.-M., S\check{s}derstr\check{s}m, M., Laherty, C. D., Torchia, J., Yang, W.-M., Brard, G., Ngo, S. D. & al., e. (1997) Nature 387, 43-46). To examine gene-specific activation, transcriptional activators are generated by fusing the zinc finger polypeptide to amino acids 413 to 489 of the herpes simplex virus VP16 protein (Sadowski, I., Ma, J., Triezenberg, S. & Ptashne, M. (1988) Nature 335, 563-564), or to an artificial tetrameric repeat of VP16's minimal activation domain, (Seipel, K., Georgiev, O. & Schaffner, W. (1992) EMBO J. 11, 4961-4968), termed VP64.

A polynucleotide of this invention as set forth above, can be operatively linked to one or more transcription modulating or regulating factors. Modulating factors such as transcription activators or transcription suppressors or repressors are well known in the art. Means for operatively linking polypeptides to such factors are also well known in the art. Exemplary and preferred such factors and their use to modulate gene expression are discussed in detail hereinafter.

In order to test the concept of using zinc finger proteins as gene-specific transcriptional regulators, six-finger proteins are fused to a number of effector domains. Transcriptional repressors are generated by attaching either of three human-derived repressor domains to the zinc finger protein. The first repressor protein is prepared using the ERF repressor domain (ERD) (Sgouras, D. N., Athanasiou, M. A., Beal, G. J., Jr., Fisher, R. J., Blair, D. G. & Mavrothalassitis, G. J. (1995) EMBO J. 14, 4781-4793), defined by amino acids 473 to 530 of the ets2 repressor factor (ERF). This domain mediates the antagonistic effect of ERF on the activity of transcription factors of the ets family. A synthetic repressor is constructed by fusion of this domain to the C-terminus of the zinc finger protein. The second repressor protein is prepared using the Krüppel-associated box (KRAB) domain (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). This repressor domain is commonly found at the N-terminus of zinc finger proteins and presumably exerts its repressive activity on TATA-dependent transcription in a distance- and orientation-independent manner (Pengue, G. & Lania, L. (1996) Proc. Natl. Acad. Sci. USA 93, 1015-1020), by interacting with the RING finger protein KAP-1 (Friedman, J. R., Fredericks, W. J., Jensen, D. E., Speicher, D. W., Huang, X.-P., Neilson, E. G. & Rauscher III, F. J. (1996) Genes & Dev. 10, 2067-2078). We utilize the KRAB domain found between amino acids 1 and 97 of the zinc finger protein KOX1 (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513). In this case an N-terminal fusion with the six-finger protein is constructed. Finally, to explore the utility of histone deacetylation for repression, amino acids 1 to 36 of the Mad mSIN3 interaction domain (SID) are fused to the N-terminus of a zinc finger protein (Ayer, D. E., Laherty, C. D., Lawrence, Q. A., Armstrong, A. P. & Eisenman, R. N. (1996) Mol. Cell. Biol. 16, 5772-5781). This small domain is found at the N-terminus of the transcription factor Mad and is responsible for mediating its transcriptional repression by interacting with mSIN3, which in turn interacts the co-repressor N-CoR and with the histone deacetylase mRPD1 (Heinzel, T., Lavinsky, R. M., Mullen, T.-M., S\check{s}derstr\check{s}m, M., Laherty, C. D., Torchia, J., Yang, W.-M., Brard, G., Ngo, S. D. & al., e. (1997) Nature 387, 43-46).

To examine gene-specific activation, transcriptional activators are generated by fusing the zinc finger protein to amino acids 413 to 489 of the herpes simplex virus VP16 protein (Sadowski, I., Ma, J., Triezenberg, S. & Ptashne, M. (1988) Nature 335, 563-564), or to an artificial tetrameric repeat of VP16's minimal activation domain, DALDDFDLDML (SEQ ID NO:36) (Seipel, K., Georgiev, O. & Schaffner, W. (1992) EMBO J. 11, 4961-4968), termed VP64.

Reporter constructs containing fragments of the erbB-2 promoter coupled to a luciferase reporter gene are generated to test the specific activities of our designed transcriptional regulators. The target reporter plasmid contains nucleotides −758 to −1 with respect to the ATG initiation codon. Promoter fragments display similar activities when transfected transiently into HeLa cells, in agreement with previous observations (Hudson, L. G., Ertl, A. P. & Gill, G. N. (1990) J. Biol. Chem. 265, 4389-4393). To test the effect of zinc finger-repressor domain fusion constructs on erbB-2 promoter activity, HeLa cells are transiently co-transfected with zinc finger expression vectors and the luciferase reporter constructs. Significant repression is observed with each construct. The utility of gene-specific polydactyl proteins to mediate activation of transcription is investigated using the same two reporter constructs.

The data herein show that zinc finger proteins capable of binding novel 9- and 18-bp DNA target sites can be rapidly prepared using pre-defined domains recognizing 5′-CNN-3′ sites. This information is sufficient for the preparation of 16⁶ or 17 million novel six-finger proteins each capable of binding 18 bp of DNA sequence. This rapid methodology for the construction of novel zinc finger proteins has advantages over the sequential generation and selection of zinc finger domains proposed by others (Greisman, H. A. & Pabo, C. O. (1997) Science 275, 657-661) and takes advantage of structural information that suggests that the potential for the target overlap problem as defined above might be avoided in proteins targeting 5′-CNN-3′ sites. Using the complex and well studied erbB-2 promoter and live human cells, the data demonstrate that these proteins, when provided with the appropriate effector domain, can be used to provoke or activate expression and to produce graded levels of repression down to the level of the background in these experiments.

### IV. POLYNUCLEOTIDES, EXPRESSION VECTORS AND TRANSFORMED CELLS

The invention includes a nucleotide sequence encoding a zinc finger-nucleotide binding polypeptide. DNA sequences encoding the zinc finger-nucleotide binding polypeptides of the invention, including native, truncated, and expanded polypeptides, can be obtained by several methods. For example, the DNA can be isolated using hybridization procedures that are well known in the art. These include, but are not limited to: (1) hybridization of probes to genomic or cDNA libraries to detect shared nucleotide sequences; (2) antibody screening of expression libraries to detect shared structural features; and (3) synthesis by the polymerase chain reaction (PCR). RNA sequences of the invention can be obtained by methods known in the art (See, for example, Current Protocols in Molecular Biology, Ausubel, et al., Eds., 1989).

The development of specific DNA sequences encoding zinc finger-nucleotide binding polypeptides of the invention can be obtained by: (1) isolation of a double-stranded DNA sequence from the genomic DNA; (2) chemical manufacture of a DNA sequence to provide the necessary codons for the polypeptide of interest; and (3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA. Of these three methods for developing specific DNA sequences for use in recombinant procedures, the isolation of genomic DNA is the least common. This is especially true when it is desirable to obtain the microbial expression of mammalian polypeptides due to the presence of introns. For obtaining zinc finger derived-DNA binding polypeptides, the synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the formation of cDNA sequences. Among the standard procedures for isolating cDNA sequences of interest is the formation of plasmid-carrying cDNA libraries which are derived from reverse transcription of mRNA which is abundant in donor cells that have a high level of genetic expression. When used in combination with polymerase chain reaction technology, even rare expression products can be clones. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucleic Acid Research 11:2325, 1983).

### V. PHARMACEUTICAL COMPOSITIONS

In another aspect, the present invention provides a pharmaceutical composition comprising a therapeutically effective amount of a zinc finger-nucleotide binding polypeptide or composition or a therapeutically effective amount of a nucleotide sequence that encodes a zinc finger-nucleotide binding polypeptide in combination with a pharmaceutically acceptable carrier.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the composition.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified. The active ingredient can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, as well as pH buffering agents and the like which enhance the effectiveness of the active ingredient.

The therapeutic pharmaceutical composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

### VI. USES

In one embodiment, a method of the invention includes a process for modulating (inhibiting or suppressing) expression of a nucleotide sequence that contains a CNN target sequence. The method includes the step of contacting the nucleotide with an effective amount of a zinc finger-nucleotide binding polypeptide of this invention that binds to the motif. In the case where the nucleotide sequence is a promoter, the method includes inhibiting the transcriptional transactivation of a promoter containing a zinc finger-DNA binding motif. The term “inhibiting” refers to the suppression of the level of activation of transcription of a structural gene operably linked to a promoter, containing a zinc finger-nucleotide binding motif, for example. In addition, the zinc finger-nucleotide binding polypeptide can bind a target within a structural gene or within an RNA sequence.

The term “effective amount” includes that amount which results in the deactivation of a previously activated promoter or that amount which results in the inactivation of a promoter containing a target nucleotide, or that amount which blocks transcription of a structural gene or translation of RNA. The amount of zinc finger derived-nucleotide binding polypeptide required is that amount necessary to either displace a native zinc finger-nucleotide binding protein in an existing protein/promoter complex, or that amount necessary to compete with the native zinc finger-nucleotide binding protein to form a complex with the promoter itself. Similarly, the amount required to block a structural gene or RNA is that amount which binds to and blocks RNA polymerase from reading through on the gene or that amount which inhibits translation, respectively. Preferably, the method is performed intracellularly. By functionally inactivating a promoter or structural gene, transcription or translation is suppressed. Delivery of an effective amount of the inhibitory protein for binding to or “contacting” the cellular nucleotide sequence containing the target sequence can be accomplished by one of the mechanisms described herein, such as by retroviral vectors or liposomes, or other methods well known in the art. The term “modulating” refers to the suppression, enhancement or induction of a function. For example, the zinc finger-nucleotide binding polypeptide of the invention can modulate a promoter sequence by binding to a target sequence within the promoter, thereby enhancing or suppressing transcription of a gene operatively linked to the promoter nucleotide sequence. Alternatively, modulation may include inhibition of transcription of a gene where the zinc finger-nucleotide binding polypeptide binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript.

The promoter region of a gene includes the regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA to RNA. In most cases the resulting RNA molecule serves as a template for synthesis of a specific protein; sometimes RNA itself is the final product.

The promoter region may be a normal cellular promoter or, for example, an onco-promoter. An onco-promoter is generally a virus-derived promoter. For example, the long terminal repeat (LTR) of retroviruses is a promoter region that may be a target for a zinc finger binding polypeptide variant of the invention. Promoters from members of the Lentivirus group, which include such pathogens as human T-cell lymphotrophic virus (HTLV) 1 and 2, or human immunodeficiency virus (HIV) 1 or 2, are examples of viral promoter regions which may be targeted for transcriptional modulation by a zinc finger binding polypeptide of the invention.

A target CNN nucleotide sequence can be located in a transcribed region of a gene or in an expressed sequence tag. A gene containing a target sequence can be a plant gene, an animal gene or a viral gene. The gene can be a eukaryotic or prokaryotic gene such as a bacterial gene. The animal gene can be a mammalian gene including a human gene. In a preferred embodiment, a method of modulating nucleotide expression is accomplished by transforming a cell that contains a target nucleotide sequence with a polynucleotide that encodes a polypeptide or composition of this invention. Preferably, the encoding polynucleotide is contained in an expression vector suitable for use in a target cell. Suitable expression vectors are well known in the art.

The CNN target exist in any combination with other target triplet sequences. That is, a particular CNN target can exist as part of an extended CNN sequence (e.g., [CNN]₂₋₁₂) or as part of any other extended sequence such as (GNN)₁₋₁₂, (ANN)₁₋₁₂, (TNN)₁₋₁₂ or (NNN)₁₋₁₂. The Examples that follow illustrate preferred embodiments of the present invention and are not limiting of the specification and claims in any way.

### EXAMPLE 1

### Construction of Zinc Finger Library and Selection Via Phase Display

Construction of the zinc finger library was based on the earlier described C7 protein ([Wu et al., (1995) PNAS 92, 344-348]; FIG. 1A, upper panel). Finger 3 recognizing the 5′-GCG-3′ subsite was replaced by a domain binding to a 5′-GAT-3′ subsite [Segal et al., (1999) Proc Natl Acad Sci USA 96(6), 2758-2763] via a overlap PCR strategy using a primer coding for finger 3 (5′-GAGGAAGTTTGCCACCAGTGGCAACCTG GTGAGGCATACCAAAATC-3′) (SEQ ID NO:31) and a pMal-specific primer (5′-GTAAAACGACGGCCAG TGCCAAGC-3′) (SEQ ID NO:32). Randomization the zinc finger library by PCR overlap extension was essentially as described [Wu et al., (1995) PNAS 92, 344-348; Segal et al., (1999) Proc Natl Acad Sci USA 96(6), 2758-2763]. The library was ligated into the phagemid vector pComb3H [Rader et al., (1997) Curr. Opin. Biotechnol. 8(4), 503-508]. Growth and precipitation of phage were performed as previously described [Barbas et al., (1991) Methods: Companion Methods Enzymol. 2(2), 119-124; Barbas et al., (1991) Proc. Natl. Acad. Sci. USA 88, 7978-7982; Segal et al., (1999) Proc Natl Acad Sci USA 96(6), 2758-2763]. Binding reactions were performed in a volume of 500 ml zinc buffer A (ZBA: 10 mM Tris, pH 7.5/90 mM KCl/lm M MgCl₂/90 mM ZnCl₂)/0.2% BSA/5 mM DTT/1% Blotto (Biorad)/20 mg double-stranded, sheared herring sperm DNA containing 100 ml precipitated phage (10¹³ colony-forming units). Phage were allowed to bind to non-biotinylated competitor oligonucleotides for 1 hr at 4° C. before the biotinylated target oligonucleotide was added. Binding continued overnight at 4° C. After incubation with 50 ml streptavidin coated magnetic beads (Dynal; blocked with 5% Blotto in ZBA) for 1 hr, beads were washed ten times with 500 ml ZBA/2% Tween 20/5 mM DTT, and once with buffer containing no Tween. Elution of bound phage was performed by incubation in 25 ml trypsin (10 mg/ml) in TBS (Tris-buffered saline) for 30 min at room temperature. Hairpin competitor oligonucleotides had the sequence 5′-GGCCGCN′N′N′ATCGAGTTTTCTCGATNN NGCGGCC-3′ (SEQ ID NO:33) (target oligonucleotides were biotinylated), where NNN represents the finger-2 subsite oligonucleotides, N′N′N′ its complementary bases. Target oligonucleotides were usually added at 72 nM in the first three rounds of selection, then decreased to 36 nM and 18 nM in the sixth and last round. As competitor a 5′-TGG-3′ finger-2 subsite oligonucleotide was used to compete with the parental clone. An equimolar mixture of 15 finger-2 5′-CNN-3′ subsites, except for the target site, respectively, and competitor mixtures of each finger-2 subsites of the type 5′-ANN-3′,5′-GNN-3′, and 5′-TNN-3′ were added in increasing amounts with each successive round of selection. Usually no specific 5′-CNN-3′ competitor mix was added in the first round.

### EXAMPLE 2

### Multitarget Specificity Assay and Gel Mobility Shift Analysis

The zinc finger-coding sequence was subcloned from pComb3H into a modified bacterial expression vector pMal-2 (New England Biolabs). After transformation into XL1-Blue (Stratagene) the zinc finger-maltose-binding protein (MBP) fusions were expressed after addition of 1 nM isopropyl b-D-thiogalactoside (IPTG). Freeze/thaw extracts of these bacterial cultures were applied in 1:2 dilutions to 96-well plates coated with streptavidin (Pierce), and were tested for DNA-binding specificity against each of the sixteen 5′-GAT CNN GCG-3′ (SEQ ID NO:34) target sites, respectively. ELISA (enzyme-linked immunosorbant assay) was performed essentially as described [Segal et al., (1999) Proc Natl Acad Sci USA 96(6), 2758-2763; Dreier et al., (2000) J. Mol. Biol. 303, 489-502]. After incubation with a mouse anti-MBP (maltose-binding protein) antibody (Sigma, 1:1000), a goat anti-mouse antibody coupled with alkaline phosphatase (Sigma, 1:1000) was applied. Detection followed by addition of alkaline phosphatase substrate (Sigma), and the OD405 was determined with SOFTMAX2.35 (Molecular Devices).

Gelshift analysis was performed with purified protein (Protein Fusion and Purification System, New England Biolabs) essentially as described.

### EXAMPLE 3

### Site-Directed Mutagenesis of Finger 2

Finger-2 mutants were constructed by PCR as described [Segal et al., (1999) Proc Natl Acad Sci USA 96(6), 2758-2763; Dreier et al., (2000) J. Mol. Biol. 303, 489-502]. As PCR template the library clone containing 5′-TGG-3′ finger 2 and 5′-GAT-3′ finger 3 was used. PCR products containing a mutagenized finger 2 and 5′-GAT-3′ finger 3 were subcloned via NsiI and SpeI restriction sites in frame with finger 1 of C7 into a modified pMal-c2 vector (New England Biolabs). Three-finger proteins were constructed by finger-2 stitchery using the SP1C framework as described [Beerli et al., (1998) Proc Natl Acad Sci USA 95(25), 14628-14633]. The proteins generated in this work contained helices recognizing 5′-GNN-3′ DNA sequences [Segal et al., (1999) Proc Natl Acad Sci USA 96(6), 2758-2763], as well as 5′-ANN-3′ and 5′-TAG-3′ helices described here. Six finger proteins were assembled via compatible XmaI and BsrFI restriction sites. Analysis of DNA-binding properties were performed from IPTG-induced freeze/thaw bacterial extracts.

### EXAMPLE 4

### General Methods

Transfection and Luciferase Assays

HeLa cells were used at a confluency of 40-60%. Cells were transfected with 160 ng reporter plasmid (pGL3-promoter constructs) and 40 ng of effector plasmid (zinc finger-effector domain fusions in pcDNA3) in 24 well plates. Cell extracts were prepared 48 hrs after transfection and measured with luciferase assay reagent (Promega) in a MicroLumat LB96P luminometer (EG & Berthold, Gaithersburg, Md.).

Retroviral Gene Targeting and Flow Cytometric Analysis

These assays were performed as described [Beerli et al., (2000) Proc Natl Acad Sci USA 97(4), 1495-1500; Beerli et al., (2000) J. Biol. Chem. 275(42), 32617-32627]. As primary antibody an ErbB-1-specific mAb EGFR (Santa Cruz), ErbB-2-specific mAb FSP77 (gift from Nancy E. Hynes; Harwerth et al., 1992) and an ErbB-3-specific mAb SGP1 (Oncogene Research Products) were used. Fluorescently labeled donkey F(ab′)2 anti-mouse IgG was used as secondary antibody (Jackson Immuno-Research).

### EXAMPLE 5

### Bacterial Extracts of pMal-Fusion Proteins for ELISA Assays

The selected zinc finger proteins were cloned into the pMal vector (New England Biolabs) for expression. The constructs were transferred into the E. coli strain XL1-Blue by electroporation and streaked on LB plates containing 50 3 g/ml carbenecillin. Four single colonies of each mutant were inoculated into 3 ml of SB media containing 50 3 g/ml carbenecillin and 1% glycose. Cultures were grown overnight at 37° C. 1.2 ml of the cultures were transformed into 20 ml of fresh SB media containing 50 3 g/ml Carbenecillin, 0.2% glycose, 90 3 g/ml ZnCl₂ and grown at 37° C. for another 2 hours. IPTG was added to a final concentration of 0.3 mM. Incubation was continued for 2 hours. The cultures were centrifuged at 4° C. for 5 minutes at 3500 rpm in a Beckman GPR centrifuge. Bacterial pellets were resuspended in 1.2 ml of Zinc Buffer A containing 5 mM fresh DTT. Protein extracts were isolated by freeze/thaw procedure using dry ice/ethanol and warm water. This procedure was repeated 6 times. Samples were centrifuged at 4° C. for 5 minutes in an Eppendorf centrifuge. The supernatant was transferred to a clean 1.5 ml centrifuge tube and used for the ELISA assays.

ELISA assays—Finger-2 variants of C7.GAT were subcloned into bacterial expression vector as fusion with maltose-binding protein (MBP) and proteins were expressed by induction with 1 mM IPTG (proteins (p) are given the name of the finger-2 subsite against which they were selected). Proteins were tested by enzyme-linked immunosorbant assay (ELISA) against each of the 16 finger-2 subsites of the type 5′-GAT CNN GCG-3′ (SEQ ID NO:34) to investigate their DNA-binding specificity.

In addition, the 5′-nucleotide recognition was analyzed by exposing zinc finger proteins to the specific target oligonucleotide and three subsites which differed only in the 5′-nucleotide of the middle triplet. For example, pCAA was tested on 5′-7AAA-3′,5′-CAA-3′, 5′-GAA-3′, and 5′-TAA-3′ subsites. Many of the tested 3-finger proteins showed exquisite DNA-binding specificity for the finger-2 subsite against they were selected. (See Table 1, below).

| TARGET | ZINC FINGER | HEPTAMER |
|---|---|---|
| CAA | SEQ ID NO: 1 | QRHNLTE |
| | SEQ ID NO: 2 | QSGNLTE |
| CAC | SEQ ID NO: 3 | NLQHLGE |
| CAG | SEQ ID NO: 4 | RADNLTE |
| | SEQ ID NO: 5 | RADNLAI |
| | SEQ ID NO: 14 | RSDHLTE |
| | SEQ ID NO: 16 | RSDHLTD |
| | SEQ ID NO: 8 | RNDTLTE |
| CAT | SEQ ID NO: 1 | QRHNLTE |
| | SEQ ID NO: 6 | NTTHLEH |
| | SEQ ID NO: 24 | TKQTLTE |
| | SEQ ID NO: 3 | NLQHLGE |
| CCA | SEQ ID NO: 6 | NTTHLEH |
| | SEQ ID NO: 25 | QSGDLTE |
| CCC | SEQ ID NO: 7 | SKKHLAE |
| CCG | SEQ ID NO: 8 | RNDTLTE |
| | SEQ ID NO: 9 | RNDTLQA |
| CCT | SEQ ID NO: 6 | NTTHLEH |
| CGA | SEQ ID NO: 10 | QSGHLTE |
| | SEQ ID NO: 11 | QLAHLKE |
| | SEQ ID NO: 12 | QRAHLTE |
| | SEQ ID NO: 17 | RSDHLTN |
| CGC | SEQ ID NO: 13 | HTGHLLE |
| CGG | SEQ ID NO: 14 | RSDHLTE |
| | SEQ ID NO: 15 | RSDKLTE |
| | SEQ ID NO: 16 | RSDHLTD |
| | SEQ ID NO: 17 | RSDHLTN |
| | SEQ ID NO: 8 | RNDTLTE |
| CGT | SEQ ID NO: 18 | SRRTCRA |
| | SEQ ID NO: 19 | QLRHLRE |
| | SEQ ID NO: 7 | SKKHLAE |
| CTA | SEQ ID NO: 20 | QRHSLTE |
| CTC | SEQ ID NO: 21 | QLAHLKE |
| | SEQ ID NO: 22 | NLQHLGE |
| CTG | SEQ ID NO: 23 | RNDALTE |
| | SEQ ID NO: 5 | RADNLAI |
| | SEQ ID NO: 8 | RNDTLTE |
| | SEQ ID NO: 14 | RSDHLTE |
| | SEQ ID NO: 9 | RNDTLQA |
| CTT | SEQ ID NO: 6 | NTTHLEH |

### EXAMPLE 6

### Gel Mobility Shift Assays

Zinc finger polypeptides linked to transcription regulating factors are purified to >90% homogeneity using the Protein Fusion and Purification System (New England Biolabs), except that ZBA/5 mM DTT is used as the column buffer. Protein purity and concentration are determined from Coomassie blue-stained 15% SDS-PAGE gels by comparison to BSA standards. Target oligonucleotides are labeled at their 5′ or 3′ ends with [³²P] and gel purified. Eleven 3-fold serial dilutions of protein are incubated in 20 μl binding reactions (1× Binding Buffer/10% glycerol/>>1 pM target oligonucleotide) for three hours at room temperature, then resolved on a 5% polyacrylamide gel in 0.5×TBE buffer. Quantitation of dried gels is performed using a Phosphorimager and ImageQuant software (Molecular Dynamics), and the K_{D} was determined by scatchard analysis.

### EXAMPLE 7

### Construction of Zinc Finger-Effector Domain Fusion Proteins

For the construction of zinc finger-effector domain fusion proteins, DNAs encoding amino acids 473 to 530 of the ets repressor factor (ERF) repressor domain (ERD) (Sgouras, D. N., Athanasiou, M. A., Beal, G. J., Jr., Fisher, R. J., Blair, D. G. & Mavrothalassitis, G. J. (1995) EMBO J. 14, 4781-4793), amino acids 1 to 97 of the KRAB domain of KOX1 (Margolin, J. F., Friedman, J. R., Meyer, W., K.-H., Vissing, H., Thiesen, H.-J. & Rauscher III, F. J. (1994) Proc. Natl. Acad. Sci. USA 91, 4509-4513), or amino acids 1 to 36 of the Mad mSIN3 interaction domain (SID) (Ayer, D. E., Laherty, C. D., Lawrence, Q. A., Armstrong, A. P. & Eisenman, R. N. (1996) Mol. Cell. Biol. 16, 5772-5781) are assembled from overlapping oligonucleotides using Taq DNA polymerase. The coding region for amino acids 413 to 489 of the VP16 transcriptional activation domain (Sadowski, I., Ma, J., Triezenberg, S. & Ptashne, M. (1988) Nature 335, 563-564) is PCR amplified from pcDNA3/C7-C7-VP16 (10). The VP64 DNA, encoding a tetrameric repeat of VP16's minimal activation domain, comprising amino acids 437 to 447 (Seipel, K., Georgiev, O. & Schaffner, W. (1992) EMBO J. 11, 4961-4968), is generated from two pairs of complementary oligonucleotides. The resulting fragments are fused to zinc finger coding regions by standard cloning procedures, such that each resulting construct contained an internal SV40 nuclear localization signal, as well as a C-terminal HA decapeptide tag. Fusion constructs are cloned in the eucaryotic expression vector pcDNA3 (Invitrogen).

### EXAMPLE 8

### Construction of Luciferase Reporter Plasmids

An erbB-2 promoter fragment comprising nucleotides −758 to −1, relative to the ATG initiation codon, is PCR amplified from human bone marrow genomic DNA with the TaqExpand DNA polymerase mix (Boehringer Mannheim) and cloned into pGL3basic (Promega), upstream of the firefly luciferase gene. A human erbB-2 promoter fragment encompassing nucleotides −1571 to −24, is excised from pSVOALD5′/erbB-2(N—N) (Hudson, L. G., Ertl, A. P. & Gill, G. N. (1990) J. Biol. Chem. 265, 4389-4393) by Hind3 digestion and suboloned into pGL3basic, upstream of the firefly luciferase gene.

### EXAMPLE 9

### Luciferase Assays

For all transfections, HeLa cells are used at a confluency of 40-60%. Typically, cells are transfected with 400 ng reporter plasmid (pGL3-promoter constructs or, as negative control, pGL3basic), 50 ng effector plasmid (zinc finger constructs in pcDNA3 or, as negative control, empty pcDNA3), and 200 ng internal standard plasmid (phrAct-bGal) in a well of a 6 well dish using the lipofectamine reagent (Gibco BRL). Cell extracts are prepared approximately 48 hours after transfection. Luciferase activity is measured with luciferase assay reagent (Promega), bGal activity with Galacto-Light (Tropix), in a MicroLumat LB96P luminometer (EG&G Berthold). Luciferase activity is normalized on bGal activity.

### EXAMPLE 10

### Regulation of the erbB-2 Gene in Hela Cells

The erbB-2 gene is targeted for imposed regulation. To regulate the native erbB-2 gene, a synthetic repressor protein and a transactivator protein are utilized (R. R. Beerli, D. J. Segal, D. Dreier, C. F. Barbas, III, Proc. Natl. Acad. Sci. USA 95, 14628 (1998)). This DNA-binding protein is constructed from 6 pre-defined and modular zinc finger domains (D. J. Segal, B. Dreier, R. R. Beerli, C. F. Barbas, III, Proc. Natl. Acad. Sci. USA 96, 2758 (1999)). The repressor protein contains the Kox-1 KRAB domain (J. F. Margolin et al., Proc. Natl. Acad. Sci. USA 91, 4509 (1994)), whereas the transactivator VP64 contains a tetrameric repeat of the minimal activation domain (K. Seipel, O. Georgiev, W. Schaffner, EMBO J. 11, 4961 (1992)) derived from the herpes simplex virus protein VP16.

A derivative of the human cervical carcinoma cell line HeLa, HeLa/tet-off, is utilized (M. Gossen and H. Bujard, Proc. Natl. Acad. Sci. USA 89, 5547 (1992)). Since HeLa cells are of epithelial origin they express ErbB-2 and are well suited for studies of erbB-2 gene targeting. HeLa/tet-off cells produce the tetracycline-controlled transactivator, allowing induction of a gene of interest under the control of a tetracycline response element (TRE) by removal of tetracycline or its derivative doxycycline (Dox) from the growth medium. We use this system to place our transcription factors under chemical control. Thus, repressor and activator plasmids are constructed and subcloned into pRevTRE (Clontech) using BamH1 and Cla1 restriction sites, and into pMX-IRES-GFP [X. Liu et al., Proc. Natl. Acad. Sci. USA 94, 10669 (1997)] using BamH1 and Not1 restriction sites. Fidelity of the PCR amplification are confirmed by sequencing), transfected into HeLa/tet-off cells, and 20 stable clones each are isolated and analyzed for Dox-dependent target gene regulation. (The contructs are transfected into the HeLa/tet-off cell line (M. Gossen and H. Bujard, Proc. Natl. Acad. Sci. USA 89, 5547 (1992)) using Lipofectamine Plus reagent (Gibco BRL). After two weeks of selection in hygromycin-containing medium, in the presence of 2 mg/ml Dox, stable clones are isolated and analyzed for Dox-dependent regulation of ErbB-2 expression. Western blots, immunoprecipitations, Northern blots, and flow cytometric analyses are carried out essentially as described [D. Graus-Porta, R. R. Beerli, N. E. Hynes, Mol. Cell. Biol. 15, 1182 (1995)]. As a read-out of erbB-2 promoter activity, ErbB-2 protein levels are initially analyzed by Western blotting. A significant fraction of these clones will show regulation of ErbB-2 expression upon removal of Dox for 4 days, i.e., downregulation of ErbB-2 in repressor clones and upregulation in activator clones. ErbB-2 protein levels are correlated with altered levels of their specific mRNA, indicating that regulation of ErbB-2 expression is a result of repression or activation of transcription.

### EXAMPLE 11

### Introduction of the Coding Regions of the E2S-KRAB, E2S-VP64, E3F-KRAB and E3F-VP64 Proteins into the Retroviral Vector pMX-IRES-GFP

In order to express the E2S-KRAB, E2S-VP64, E3F-KRAB and E3F-VP64 proteins (See Table 2, below) in several cell lines, their coding regions were introduced into the retroviral vector pMX-IRES-GFP.

| DNA Target e2t | | | | | | |
|---|---|---|---|---|---|---|
| 5′→3′ | CAA | CGA | AGT | CTG | GGA | GTC |
| Zinc Finger Sequence | QRHNLTE | QLAHLKE | HRTTLTN | RNDALTE | QRAHLER | DPGALVR |
| E2T | | | | | | |
| SEQ ID NO: | 1 | 11 | 35 | 23 | 36 | 37 |
| DNA Target e2s | | | | | | |
| 5′→3′ | CGG | GGG | GCT | CCC | CTG | GTT |
| Zinc Finger Sequence | RSDHLTE | RSDKLVR | TSGELVR | SKKHLAE | RNDALTE | TSGSLVR |
| E2S | | | | | | |
| SEQ ID NO: | 14 | 38 | 39 | 7 | 23 | 39 |
| DNA Target e3f | | | | | | |
| 5′→3′ | AGG | GGC | CCC | CGG | GCC | GGA |
| Zinc Finger Sequence | RSDHLTN | DPGHLVR | SKKHLAE | RSDHLTE | DCRDLAR | QRAHLER |
| E3F | | | | | | |
| SEQ ID NO: | 40 | 41 | 7 | 14 | 42 | 36 |

The sequences of these constructs were selected to bind to specific regions of the ErbB-2 or ErbB-3 promoters (See Table 2). The coding regions were PCR amplified from pcDNA3-based expression plasmids (R. R. Beerli, D. J. Segal, B. Dreier, C. F. Barbas, III, Proc. Natl. Acad. Sci. USA 95, 14628 (1998)) and subcloned into pRevTRE (Clontech) using BamH1 and Cla1 restriction sites, and into pMX-IRES-GFP [X. Liu et al., Proc. Natl. Acad. Sci. USA 94, 10669 (1997)] using BamH1 and Not1 restriction sites. Fidelity of the PCR amplification was confirmed by sequencing. This vector expresses a single bicistronic message for the translation of the zinc finger protein and, from an internal ribosome-entry site (IRES), the green fluorescent protein (GFP). Since both coding regions share the same mRNA, their expression is physically linked to one another and GFP expression is an indicator of zinc finger expression. Virus prepared from these plasmids was then used to infect the human carcinoma cell line A431.

### EXAMPLE 12

### Regulation of ErbB-2 and ErbB-3 Gene Expression

Plasmids from Example 11 were transiently transfected into the amphotropic packaging cell line Phoenix Ampho using Lipofectamine Plus (Gibco BRL) and, two days later, culture supernatants were used for infection of target cells in the presence of 8 mg/ml polybrene. Three days after infection, cells were harvested for analysis. Three days after infection, ErbB-2 and ErbB-3 expression was measured by flow cytometry. The results show that E2S-KRAB and E2S-VP64 compositions inhibited and enhanced ErbB-2 gene expression, respectively. The data also show that E3F-KRAB and E3F-VP64 compositions inhibited and enhanced ErbB-2 gene expression, respectively.

The human erbB-2 and erbB-3 genes were chosen as model targets for the development of zinc finger-based transcriptional switches. Members of the ErbB receptor family play important roles in the development of human malignancies. In particular, erbB-2 is overexpressed as a result of gene amplification and/or transcriptional deregulation in a high percentage of human adenocarcinomas arising at numerous sites, including breast, ovary, lung, stomach, and salivary gland (Hynes, N. E. & Stern, D. F. (1994) Biochim. Biophys. Acta 1198, 165-184). Increased expression of ErbB-2 leads to constitutive activation of its intrinsic tyrosine kinase, and has been shown to cause the transformation of cultured cells. Numerous clinical studies have shown that patients bearing tumors with elevated ErbB-2 expression levels have a poorer prognosis (Hynes, N. E. & Stern, D. F. (1994) Biochim. Biophys. Acta 1198, 165-184). In addition to its involvement in human cancer, erbB-2 plays important biological roles, both in the adult and during embryonal development of mammals (Hynes, N. E. & Stem, D. P. (1994) Biochim. Biophys. Acta 1198, 165-184, Altiok, N., Bessereau, J.-L. & Changeux, J.-P. (1995) EMBO J. 14, 4258-4266, Lee, K.-F., Simon, H., Chen, H., Bates, 13., Hung, M.-C. & Hauser, C. (1995) Nature 378, 394-398).

The erbB-2 promoter therefore represents an interesting test case for the development of artificial transcriptional regulators. This promoter has been characterized in detail and has been shown to be relatively complex, containing both a TATA-dependent and a TATA-independent transcriptional initiation site (Ishii, S., Imamoto, F., Yamanashi, Y., Toyoshima, K. & Yamamoto, T. (1987) Proc. Natl. Acad. Sci. USA 84, 437-44378). Whereas early studies showed that polydactyl proteins could act as transcriptional regulators that specifically activate or repress transcription, these proteins bound upstream of an artificial promoter to six tandem repeats of the proteins binding site (Liu, Q., Segal, D. J., Ghiara, J. B. & Barbas III, C. F. (1997) Proc. Natl. Acad. Sci. USA 94, 5525-5530). Furthermore, this study utilized polydactyl proteins that were not modified in their binding specificity. Herein, we tested the efficacy of polydactyl proteins assembled from predefined building blocks to bind a single site in the native erbB-2 and erbB-3 promoter.

For generating polydactyl proteins with desired DNA-binding specificity, the present studies have focused on the assembly of predefined zinc finger domains, which contrasts the sequential selection strategy proposed by Greisman and Pabo (Greisman, H. A. & Pabo, C. O. (1997) Science 275, 657-661). Such a strategy would require the sequential generation and selection of six zinc finger libraries for each required protein, making this experimental approach inaccessible to most laboratories and extremely time-consuming to all. Further, since it is difficult to apply specific negative selection against binding alternative sequences in this strategy, proteins may result that are relatively unspecific as was recently reported (Kim, J.-S. & Pabo, C. O. (1997) J. Biol. Chem. 272, 29795-29800).

The general utility of two different strategies for generating three-finger proteins recognizing 18 bp of DNA sequence was investigated. Each strategy was based on the modular nature of the zinc finger domain, and takes advantage of a family of zinc finger domains recognizing triplets of the 5′-NNN-3′. Three six-finger proteins recognizing halfsites erbB-2 or erbB-3 target sites were generated in the first strategy by fusing the pre-defined finger 2 (F2) domain variants together using a PCR assembly strategy.

The affinity of each of the proteins for its target was determined by electrophoretic mobility-shift assays. These studies demonstrated that the zinc finger peptides have affinities comparable to Zif268 and other natural transcription factors.

The affinity of each protein for the DNA target site is determined by gel-shift analysis.

1-74. (canceled)

75. A non-naturally occurring zinc finger nucleotide binding polypeptide comprising at least three zinc finger nucleotide binding regions, wherein at least one binding region is specific for 5′-CNN-3′ and comprises QS/RG/H/AH/N/D/SLTE (SEQ ID NO:54), N/HL/TG/QHLL/GE (SEQ ID NO:55) or RA/S/NDH/T/A/K/NLTE (SEQ ID NO:56).

76. The polypeptide of claim 75 which binds to the triplet CNA, wherein N=A, C, G or T.

77. The polypeptide of claim 75 which binds to the triplet CNG, wherein N=A, C, G or T.

78. The polypeptide of claim 75 which binds to the triplet CNC, wherein N=A, C, G or T.

79. The polypeptide of claim 75 wherein the at least one binding region has the amino acid sequence of any of SEQ ID NOs: 1-4, 8, 10, 13, 14, 15, 20, 22, 23, and 25.

80. The polypeptide of claim 75 where the zinc finger binding regions are operatively linked to one or more transcription regulating factors.

81. The polypeptide of claim 80 wherein the transcription regulating factor is a repressor of transcription.

82. The polypeptide of claim 80 wherein the transcription regulating factor is an activator of transcription.

83. A peptide composition comprising a plurality of the polypeptides of claim 75, wherein the polypeptides are operatively linked to each other.

84. The peptide composition of claim 83 wherein the plurality is operatively linked via a flexible peptide linker of from 5 to 15 amino acid residues.

85. The peptide composition of claim 84 wherein the flexible peptide linker has the amino acid residue sequence of SEQ ID NO: 30.

86. The peptide composition of claim 83 wherein a plurality is from 2 to 12.

87. The peptide composition of claim 83 wherein a plurality is from 2 to 6.

88. The peptide composition of claim 87 that binds to a nucleotide sequence that comprises a sequence having 5′-(CNA)ₙ-3′, where N is A, C, G or T and n is 2 to 12.

89. The peptide composition of claim 87 that binds to a nucleotide sequence that comprises a sequence of the formula 5′-(CNC)ₙ-3′, where N is A, C, G or T and n is 2 to 12.

90. The peptide composition of claim 83 that binds to a nucleotide sequence that comprises a sequence of the formula 5′-(CNG)ₙ-3′, where N is A, C, G or T and n is 2 To 12.

91. The peptide composition of claim 88, 89 or 90 that binds to a nucleotide sequence with a K_{D} of from 1 fM to 10 μM.

92. A polynucleotide encoding the polypeptide of claim 75.

93. A process of regulating expression of a nucleotide sequence that contains the sequence 5′-(CNN)ₙ-3′, where n is 2 to 12, the process comprising exposing the nucleotide sequence to an effective amount of the composition of claim 83.


SEQUENCE LISTING (56 sequences)

SEQ ID NO: 1 - protein, 7 aa, Artificial Sequence
  Description of Artificial Sequence Synthetic zinc finger domain
QRHNLTE

SEQ ID NO: 2 - protein, 7 aa, Artificial Sequence
  Description of Artificial Sequence Synthetic zinc finger domain
QSGNLTE

SEQ ID NO: 3 - protein, 7 aa, Artificial Sequence
  Description of Artificial Sequence Synthetic zinc finger domain
NLQHLGE

SEQ ID NO: 4 - protein, 7 aa, Artificial Sequence
  Description of Artificial Sequence Synthetic zinc finger domain
RADNLTE

SEQ ID NO: 5 - protein, 7 aa, Artificial Sequence
  Description of Artificial Sequence Synthetic zinc finger domain
RADNLAI

SEQ ID NO: 6 - protein, 7 aa, Artificial Sequence
  Description of Artificial Sequence Synthetic zinc finger domain
NTTHLEH

SEQ ID NO: 7 - protein, 7 aa, Artificial Sequence
  Description of Artificial Sequence Synthetic zinc finger domain
SKKHLAE

SEQ ID NO: 8 - protein, 7 aa, Artificial Sequence
  Description of Artificial Sequence Synthetic zinc finger domain
RNDTLTE

SEQ ID NO: 9 - protein, 7 aa, Artificial Sequence
  Description of Artificial Sequence Synthetic zinc finger domain
RNDTLQA

SEQ ID NO: 10 - protein, 7 aa, Artificial Sequence
  Description of Artificial Sequence Synthetic zinc finger domain
QSGHLTE

SEQ ID NO: 11 - protein, 7 aa, Artificial Sequence
  Description of Artificial Sequence Synthetic zinc finger domain
QLAHLKE

SEQ ID NO: 12 - protein, 7 aa, Artificial Sequence
  Description of Artificial Sequence Synthetic zinc finger domain
QRAHLTE

SEQ ID NO: 13 - protein, 7 aa, Artificial Sequence
  Description of Artificial Sequence Synthetic zinc finger domain
HTGHLLE

SEQ ID NO: 14 - protein, 7 aa, Artificial Sequence
  Description of Artificial Sequence Synthetic zinc finger domain
RSDHLTE

SEQ ID NO: 15 - protein, 7 aa, Artificial Sequence
  Description of Artificial Sequence Synthetic zinc finger domain
RSDKLTE

SEQ ID NO: 16 - protein, 7 aa, Artificial Sequence
  Description of Artificial Sequence Synthetic zinc finger domain
RSDHLTD

SEQ ID NO: 17 - protein, 7 aa, Artificial Sequence
  Description of Artificial Sequence Synthetic zinc finger domain
RSDHLTN

SEQ ID NO: 18 - protein, 7 aa, Artificial Sequence
  Description of Artificial Sequence Synthetic zinc finger domain
SRRTCRA

SEQ ID NO: 19 - protein, 7 aa, Artificial Sequence
  Description of Artificial Sequence Synthetic zinc finger domain
QLRHLRE

SEQ ID NO: 20 - protein, 7 aa, Artificial Sequence
  Description of Artificial Sequence Synthetic zinc finger domain
QRHSLTE

SEQ ID NO: 21 - protein, 7 aa, Artificial Sequence
  Description of Artificial Sequence Synthetic zinc finger domain
QLAHLKE

SEQ ID NO: 22 - protein, 7 aa, Artificial Sequence
  Description of Artificial Sequence Synthetic zinc finger domain
NLQHLGE

SEQ ID NO: 23 - protein, 7 aa, Artificial Sequence
  Description of Artificial Sequence Synthetic zinc finger domain
RNDALTE

SEQ ID NO: 24 - protein, 7 aa, Artificial Sequence
  Description of Artificial Sequence Synthetic zinc finger domain
TKQTLTE

SEQ ID NO: 25 - protein, 7 aa, Artificial Sequence
  Description of Artificial Sequence Synthetic zinc finger domain
QSGDLTE

SEQ ID NO: 26 - protein, 7 aa, Artificial Sequence
  Description of Artificial Sequence Synthetic zinc finger domain
RSDELKR

SEQ ID NO: 27 - protein, 7 aa, Artificial Sequence
  Description of Artificial Sequence Synthetic zinc finger domain
RSDERKR

SEQ ID NO: 28 - protein, 7 aa, Artificial Sequence
  Description of Artificial Sequence Synthetic zinc finger domain
TSGNLVR

SEQ ID NO: 29 - DNA, 9 bp, Artificial Sequence
  Description of Artificial Sequence Synthetic oligonucleotide
gatcnngcg

SEQ ID NO: 30 - protein, 5 aa, Artificial Sequence
  Description of Artificial Sequence Synthetic linker peptide
TGEKP

SEQ ID NO: 31 - DNA, 46 bp, Artificial Sequence
  Description of Artificial Sequence Synthetic oligonucleotide
gaggaagtttgccaccagtggcaacctggtgaggcataccaaaatc

SEQ ID NO: 32 - DNA, 24 bp, Artificial Sequence
  Description of Artificial Sequence Synthetic oligonucleotide
gtaaaacgacggccagtgccaagc

SEQ ID NO: 33 - DNA, 34 bp, Artificial Sequence
  Description of Artificial Sequence Synthetic oligonucleotide
ggccgcnnnatcgagttttctcgatnnngcggcc

SEQ ID NO: 34 - DNA, 9 bp, Artificial Sequence
  Description of Artificial Sequence Synthetic oligonucleotide
gatcnngcg

SEQ ID NO: 35 - protein, 7 aa, Artificial Sequence
  Description of Artificial Sequence Synthetic zinc finger domain
HRTTLTN

SEQ ID NO: 36 - protein, 7 aa, Artificial Sequence
  Description of Artificial Sequence Synthetic zinc finger domain
QRAHLER

SEQ ID NO: 37 - protein, 7 aa, Artificial Sequence
  Description of Artificial Sequence Synthetic zinc finger domain
DPGALVR

SEQ ID NO: 38 - protein, 7 aa, Artificial Sequence
  Description of Artificial Sequence Synthetic zinc finger domain
RSDKLVR

SEQ ID NO: 39 - protein, 7 aa, Artificial Sequence
  Description of Artificial Sequence Synthetic zinc finger domain
TSGELVR

SEQ ID NO: 40 - protein, 7 aa, Artificial Sequence
  Description of Artificial Sequence Synthetic zinc finger domain
RSDHLTN

SEQ ID NO: 41 - protein, 7 aa, Artificial Sequence
  Description of Artificial Sequence Synthetic zinc finger domain
DPGHLVR

SEQ ID NO: 42 - protein, 7 aa, Artificial Sequence
  Description of Artificial Sequence Synthetic zinc finger domain
DCRDLAR

SEQ ID NO: 43 - protein, 11 aa, Artificial Sequence
  Description of Artificial Sequence Synthetic peptide
DALDDFDLDML

SEQ ID NO: 44 - protein, 7 aa, Artificial Sequence
  Description of Artificial Sequence Synthetic zinc finger domain
KSADLKR

SEQ ID NO: 45 - protein, 7 aa, Artificial Sequence
  Description of Artificial Sequence Synthetic zinc finger domain
RSDHLTT

SEQ ID NO: 46 - DNA, 9 bp, Artificial Sequence
  Description of Artificial Sequence Synthetic oligonucleotide
gcgtgggcg

SEQ ID NO: 47 - DNA, 9 bp, Artificial Sequence
  Description of Artificial Sequence Synthetic oligonucleotide
gattgggcg

SEQ ID NO: 48 - protein, 7 aa, Artificial Sequence
  Description of Artificial Sequence Synthetic zinc finger domain
TSGSLVR

SEQ ID NO: 49 - DNA, 36 bp, Artificial Sequence
  Description of Artificial Sequence Synthetic nucleotide target motif
cnncnncnncnncnncnncnncnncnncnncnncnn

SEQ ID NO: 50 - DNA, 18 bp, Artificial Sequence
  Description of Artificial Sequence Synthetic nucleotide target motif
cnncnncnncnncnncnn

SEQ ID NO: 51 - DNA, 36 bp, Artificial Sequence
  Description of Artificial Sequence Synthetic nucleotide target motif
gnngnngnngnngnngnngnngnngnngnngnngnn

SEQ ID NO: 52 - DNA, 36 bp, Artificial Sequence
  Description of Artificial Sequence Synthetic nucleotide target motif
annannannannannannannannannannannann

SEQ ID NO: 53 - DNA, 36 bp, Artificial Sequence
  Description of Artificial Sequence Synthetic nucleotide target motif
tnntnntnntnntnntnntnntnntnntnntnntnn

SEQ ID NO: 54 - protein, 7 aa, Artificial Sequence
  Description of Artificial Sequence Synthetic zinc finger domain
QXXXLTE

SEQ ID NO: 55 - protein, 7 aa, Artificial Sequence
  Description of Artificial Sequence Synthetic zinc finger domain
XXXHLXE

SEQ ID NO: 56 - protein, 7 aa, Artificial Sequence
  Description of Artificial Sequence Synthetic zinc finger domain
RXDXLTE